(19) United States
(12) Patent Application Publication
(10) Pub. No.: US 2014/0178512 A1
(43) Pub. Date: Jun. 26, 2014

(54) IN SITU FORMATION OF A FILLER

(71) Applicants: Kevin Kiehm, Telgte (DE); Bernhard Hauptmeier, Gelnhausen (DE); Peter Boderke, Schwalbach (DE)

(72) Inventors: Kevin Kiehm, Telgte (DE); Bernhard Hauptmeier, Gelnhausen (DE); Peter Boderke, Schwalbach (DE)

(73) Assignee: MERZ PHARMA GMBh & CO. KGaA, Frankfurt am Main (DE)

(21) Appl. No.: 14/000,480

(22) PCT Filed: Feb. 17, 2012
(86) PCT No.: PCT/EP12/00718

§ 371 Date: Feb. 13, 2014

Related U.S. Application Data
(60) Provisional application No. 61/445,077, filed on Feb. 22, 2011.

(30) Foreign Application Priority Data
Feb. 22, 2011 (EP) 110010 468.5

(51) Int. Cl. A61L 27/20

(52) U.S. Cl. CPC A61L 27/20
USPC 424/780; 514/54; 536/2; 536/114

(57) ABSTRACT

The present invention pertains to a method for the in situ preparation of a filler, comprising the step of applying a polyanionic biopolymer solution at an application site below the dermis of a patient for restoring volume at said application site, wherein the polyanionic biopolymer is selected from pectin and gellan. Furthermore, the present invention pertains to a kit comprising (i) a polyanionic biopolymer solution; and (ii) a solution comprising at least one divalent cation.

### FIELD OF THE INVENTION

The present invention pertains to a method for the in situ preparation of a filler, comprising the step of applying a polyanionic biopolymer solution at an application site below the dermis of a patient for restoring volume at said application site. Furthermore, the present invention pertains to a kit comprising (i) a polyanionic biopolymer solution; and (ii) a solution comprising at least one divalent cation.

### BACKGROUND OF THE INVENTION

Treatment with fillers is known since 1980s. Today's most preferred fillers can be classified as hyaluronic acid-based fillers (Hylaform®, Hylaform® Plus, Restylane®, Perlane®, Juvederm®, Juvederm® Ultra, Juvederm® Ultra Plus, Puragen®, Puragen® Plus, Matridur®), collagen based fillers (Zyderm® I, Zyderm® II, Zyplast Atelocollagen®, CosmoDerm® I, CosmoDerm® II, Resoplast®) and alginate based fillers (e.g. Novabel®) as described in DE 10 2004 019 241.

Collagen is a natural protein of connective tissue. However, some people suffer from allergic reactions to collagen and thus, an allergy test is always suggested by the practitioner prior to injection of fillers comprising collagen. Hyaluronic acid is a polysaccharide and is naturally found in many tissues of the body. The unfavorable effect of fillers comprising hyaluronic acid is the need for multiple injections for an observable effect. Thereby swellings can occur, which decay in about 1-3 days.

The use of alginate as filler is known from DE 10 2004 019 241. However, DE 10 2004 019 241 suggests for the long-lasting effect of the cross-linked alginate particles the use of barium (paragraph [0031]).

In all these approaches, biopolymer gels and/or biopolymer beads are produced in a first step in vitro, and are then transferred to the region of the body to be treated. Thus, stability of the gel or beads and syringeability are crucial factors, which have not been resolved for all biopolymers and all indications in a fully satisfactory way.

EP 2 082 755 suggests the use of non-crosslinked alginate solutions for the in situ formation of a crosslinked alginate-based filler. However, such alginate-based fillers suffer from the disadvantages that the non-crosslinked alginate solutions exhibit high viscosities and low gelling propensities under physiological conditions (see Examples 3 and 4 below).

Therefore, there still remains a need in the art for more satisfactory methods of the application of fillers with less risk, less cost and less complications for patients and a long-lasting effect.

### OBJECTS OF THE INVENTION

Accordingly, in view of the problems of the prior art, the object of the present invention is to provide a novel method for applying a filler, which is to be applied below the dermis, thereby leaving no scar, rapidly restoring volume at application site and sustaining the volume augmentation, and which does not contain collagen, which can cause allergic reactions, thereby not requiring pre-testing, such as allergic skin testing. Furthermore, collagen is derived from animal tissue with the risk of transmission of viruses. It is also important that the filler remain evenly distributed after the injection to avoid palpable mass after the carrier is resorbed in the body. Thus, it is an object of the present invention to provide a novel filler application method exhibiting a long-lasting effect and much less side effects.

Another objective of the present invention is to provide a novel application method for a filler composition, which, unlike conventional fillers, which contain collagen or hyaluronic acid as a major component, is not easily degraded by human enzymes or absorbed in the body, thus ensuring stable longer-lasting volume augmentation, and is cheaper than conventional fillers.

Another objective of the present invention is to provide means for a convenient and simple application of the filler by physicians. Conventional fillers are viscous gel formulations and thus needles with larger diameters have to be used resulting in more pain for the patient or the necessity of greater extrusion forces to eject the filler into the tissue.

### SUMMARY OF THE INVENTION

These and other objects are solved by a method for the in situ preparation of a filler, comprising the step of applying a polyanionic biopolymer solution at an application site below the dermis of a patient for restoring volume at the application site, wherein the polyanionic biopolymer is selected from pectin and gellan.

In certain embodiments, the filler is formed in situ as a biopolymer gel by cross-linking the polyanionic biopolymer with divalent calcium ions present in the extracellular subdermal space in a concentration of approximately 2.5 mmol/l.

In one embodiment of the instant invention, the method further comprises the step of applying a solution comprising at least one divalent cation to the application site before or after the application of the polymer solution.

In another embodiment, the at least one divalent cation is taken from the group of barium, zinc, copper, calcium and magnesium, or a mixture thereof.

In one embodiment of the instant invention, at least one trivalent cation is used, which is taken from the group of aluminum and iron, or a mixture thereof.

In one embodiment of the present invention, the polyanionic biopolymer employed in the method provided in the present invention is pectin.

In one embodiment of the instant invention, the pectin has a degree of amidation from about 0% to about 60%.

In a further embodiment of the instant invention, the pectin has a degree of esterification from about 0% to about 75%.

In another embodiment of the instant invention, the pectin has a molecular weight distribution from about 50 kDa to about 5000 kDa.

In certain embodiments, the pectin comprises a content of more than about 60% galacturonic acid.

In certain embodiments, where the polyanionic biopolymer is pectin, the divalent cation is taken from the group of barium, copper, zinc, and calcium, or a mixture thereof.

In certain other embodiments of the present invention, the polyanionic biopolymer employed in the method provided in the present invention is gellan.

In one embodiment, the gellan has a molecular weight distribution from about 50 kDa to about 5000 kDa.

In certain embodiments, where the polyanionic biopolymer is gellan, the divalent cation is taken from the group of copper, zinc, and calcium, or a mixture thereof.

The polyanionic biopolymer solution and/or the solution comprising at least one divalent cation according to the present invention may further comprise one or more active pharmaceutical ingredients selected from the group of anesthetics, analgesics, anti-microbials, anti-inflammatory drugs, growth factors, hormones, cosmeceuticals, vitamins, nutrients, stimulants, steroids, vasoconstrictors, anti-thrombotic agents, anti-coagulation agents, tranquilizers, muscle relaxants, antifungals, lipolytic agents and biorejunevation agents.

The polyanionic biopolymer solution and/or the solution comprising at least one divalent cation according to the instant invention may further comprise one or more pharmaceutical excipients selected from antioxidants, viscosity enhancers/modifiers, hydrating agents, bulking substances, tonicity agents, preservatives and surface active agents, or a mixture thereof.

The polyanionic biopolymer solution and/or said solution comprising at least one divalent cation provided in the present invention may further comprise a polysaccharide.

In one embodiment, the polysaccharide is hyaluronic acid and/or salts thereof.

In one embodiment, the present invention provides a method for the in situ preparation of a filler for aesthetic purposes.

In a particular embodiment, the present invention provides a method for the in situ preparation of a dermal filler, particularly for a non-medical treatment and/or use of a dermal filler.

In a particular embodiment, the dermal filler according to the instant invention is for the treatment of, or for the use in the treatment of, wrinkles and/or folds.

In another embodiment, the polyanionic biopolymer as defined according to the instant invention is used for the treatment of, or for the use in the treatment of, a medical condition, including lipoatrophy, vocal fold insufficiency, gastroesophageal reflux disease (GERD), urine incontinence, vesico ureteral reflux (VUR), or a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles and acne scars.

In another embodiment of the present invention, the polyanionic biopolymer as defined according to the instant invention is used for the treatment of, or for the use in the treatment of, acne scars, such as by filling areas of acne scars.

The present invention further pertains to a method of treating a medical condition, including lipoatrophy, vocal fold insufficiency, gastroesophageal reflux disease (GERD), urine incontinence, vesico ureteral reflux (VUR), or a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles and acne scars, wherein said method comprises a step of administering a polyanionic biopolymer as defined in the present invention to a patient in need thereof

The present invention further pertains to a method of using a polyanionic biopolymer as defined according to the present invention in plastic, cosmetic, dental or general surgery, in ophthalmology, in orthopedics, as products for preventing tissue adhesions, or in urology, wherein said method comprises a step of administering the polyanionic biopolymer as defined in the present invention to a patient in need thereof.

In one further embodiment, the concentration of the polyanionic biopolymer is between 0.1 and 5 wt-% relative to the total weight of the solution.

In one further embodiment, where the method further comprises the step of applying a solution comprising at least one divalent cation to the application site before or after the application of the polymer solution, the amount of the at least one divalent cation is calculated in a way that up to 0.5 divalent cations, particularly between 0.4 and 0.5 divalent cations, per 1 carboxylate group in the polyanionic biopolymer are applied to the application site.

In one embodiment, the pH of the polyanionic biopolymer solution and/or the solution containing divalent cations is adjusted to a value between 7.0 and 7.8, particularly between 7.2 and 7.6, more particularly the pH value is 7.4.

In another embodiment, the viscosity of the polyanionic biopolymer solution is in the range from about 10 mPa*s to about 500 mPa*s measured by the falling ball viscometer.

In another embodiment, the in situ gelling effect determined by the change of viscosity after the in situ cross-linking is an increase of viscosity at least more than 50% of the initial viscosity of the polymer solution, more particular at least more than 100% of the initial viscosity and most particular at least more than 200% of the initial viscosity (see Example 3 below).

In another embodiment, the syringeability determined by the extrusion force of the polymer solution through a 27G needle is in the range of 1-30 N, particular between 1-20 N and more particular the extrusion force of the polymer solution through a 30G needle is between 1-25 N.

Further, the present invention pertains to a kit comprising (i) a polyanionic biopolymer solution as defined according to the present invention; and (ii) a solution comprising at least one divalent cation as defined according to the present invention.

Further, the present invention pertains to an injection device comprising a polyanionic biopolymer solution as defined according to the present invention. The injection device could be a prefilled syringe, a microneedle device or an electronic injection device.

### DETAILED DESCRIPTION OF THE INVENTION

The present invention relates to a method for the in situ preparation of a filler, comprising the step of applying a polyanionic biopolymer solution at an application site below the dermis of a patient for restoring volume at the application site, wherein the polyanionic biopolymer is selected from pectin and gellan.

The formation of the filler starting from the polyanionic biopolymer solution, which contains the biopolymer exclusively or predominantly in a non-crosslinked state, is the result of an in vivo crosslinking. This in vivo crosslinking may be either achieved by calcium ions that are present in the patient or by calcium ions that are applied as a solution of calcium ions parallel, before or after the application of the polyanionic biopolymer solution, or a combination of both routes.

The term “filler” as used in the instant invention relates to compositions, which are administered for augmentation, repair or strengthening of tissue, or for filling a bodily cavity, in a mammal. The term “mammal” as used herein refers to a human or an animal taken from the list of farm animals like horses, cattle, pig, camel, chicken, turkey, or pets like dog, or cat.

The term “biopolymer” as used in the present invention relates to polymers of natural origin or synthetic or biotechnological derivatives of such natural polymers. The term “polymer” as used in the instant invention relates to macromolecules composed of repeating structural units connected by chemical bonds. The term “polyanionic biopolymer” as used in the present invention relates to a biopolymer, wherein some or all of the repeating structural units carry, or can result in the formation of, a negatively charged functional group, such as a carboxylate, sulfonate, amidate or phosphate moiety. In the present invention, the polyanionic biopolymer is taken from the list of: pectin and gellan.

In one embodiment of the instant invention, the method further comprises the step of applying a solution comprising at least one divalent cation to the application site.

In one embodiment, the at least one divalent cation is taken form the group of barium, zinc, copper, calcium and magnesium, or a mixture thereof.

In another embodiment, a trivalent cation is used, which is taken from the group of aluminum and iron.

In another embodiment, a mixture of one or more di- and/or trivalent cations is used, which are taken from the group barium, zinc, copper, calcium, magnesium, aluminum and iron.

In certain embodiments of the present invention, the polyanionic biopolymer employed in the method provided in the present invention is pectin.

The term “pectin” as used in the present invention relates to a heteropolysaccharide comprising a linear chain of α-(1-4)-linked D-galacturonic acid, which forms the pectin backbone as homogalacturonan and/or salts thereof.

In one embodiment of the instant invention, the pectin has a degree of amidation from about 0% to about 30%.

In one further embodiment of the instant invention, the pectin has a degree of esterification from about 0% to about 75%.

In another embodiment of the instant invention, the pectin has a molecular weight distribution from about 50 kDa to about 5000 kDa.

In certain embodiments, the pectin comprises a content of more than about 60% galacturonic acid.

Non-limiting examples for the pectin which may be employed in the filler provided in the instant invention are citrus pectin, apple pectin, grapefruit pectin, carrot pectin and pectins manufactured by biotechnological and/or enzymatic methods, grape pectin, plum pectin, pear pectin, cherry pectin, currant pectin.

In certain embodiments, where the polyanionic biopolymer is pectin, the divalent cation is taken from the group of barium, zinc, copper and calcium, or a mixture thereof.

In certain embodiments, the polyanionic biopolymer employed in the method provided in the present invention is gellan. Gellan gum is a high molecular weight polysaccharide comprising a tetrasaccharide repeating unit of rhamnose, guluronic acid and glucose units. It may contain acyl (glyceryl and acetyl) groups as the O-glycosidically linked esters.

The term “gellan” or “gellan gum” as used in the present invention are used interchangeably and refer to a water-soluble polysaccharide produced by Sphingomonas elodea or Sphingomonas paucimobilis (ATCC 31461, E2(DSM 6314), NK2000, GS1) and/or salts thereof.

In certain embodiments, where the polyanionic biopolymer is gellan, the divalent cation is taken from the group of copper, zinc and calcium, or a mixture thereof.

In one embodiment, the gellan has a molecular weight distribution from about 50 to about 5000 kDa.

A non-limiting example for a gellan, which may be employed in the filler provided in the instant invention, is gelrite® and/or gelzan®.

The term “molecular weight distribution” as used in the present invention refers to a range or distribution of the molecular weights of a population of molecules, which are not homogeneous with respect to molecular size and weight, and which thus can best be described by a range of molecular weights characterized by a lower and an upper limit, where such range covers about at least 60%, particularly at least 70%, more particularly at least 80%, and most particularly at least 90% of all molecular weights present in a given sample.

The term “about” as used in the present invention refers to a 10% deviation from the value it is attached to.

According to the instant invention the polyanionic biopolymer solution and/or the solution comprising at least one divalent cation may comprise a medium in which the polymer is dissolved or dispersed. Said medium may be sterile water, phosphate-buffer saline (PBS), ringer solution, isotonic saline solution (0.9%), trometamol, citrate, carbonate, acetate, borate, amino acid, diethylamine, glucono delta lactone, glycine, lactate, histidine, maleic, methanesulfonic, monoethanolamine, tartrate buffer of choice or any combination thereof.

The polyanionic biopolymer solution and/or the solution comprising at least one divalent cation as claimed in the instant invention may further comprise one or more active pharmaceutical ingredients selected from the group of anesthetics, analgesics, anti-microbials, anti-inflammatory drugs, growth factors, hormones, cosmeceuticals, vitamins, nutrients, stimulants, steroids, vasoconstrictors, anti-thrombotic agents, anti-coagulation agents, tranquilizers, muscle relaxants, antifungals, lipolytic agents and biorejunevation agents.

The term “active pharmaceutical ingredient” refers to all structures, which are pharmacologically active, thus resulting in a pharmacological effect in mammal and all known chemical forms thereof. Examples are, but not limited to, conjugates, isomers, esters, derivatives, metabolites, residues, salts or prodrugs thereof.

Anesthetics may be, but are not limited to, local anesthetics based on esters (Procaine, Benzocaine, Chloroprocaine, Cocaine, Cyclomethycaine, Dimethodcaine, Larocaine, Propoxycaine, Proparacaine, Tretracaine) or local anesthetics based on amides (Lidocaine, Articaine, Bupivacaine, Carticaine, Cinchocaine, Etidocaine, Levobupivacaine, Mepivacaine, Piperocaine, Prilocaine, Ropivacaine, Trimecaine). A suitable concentration for the anesthetic is from about 0.01% to 6% based on the total weight of the composition and the agent selected.

Analgesics may be, but are not limited to, paracetamol, ibuprofen, diclofenac, naproxen, aspirin, celecoxib, etoricoxib, lumiracoxib, parecoxib, rofecoxib, valdecoxib, nimesulid, oxicams, such as piroxicam, isoxicam, tonexicam, sudoxicam, and CP-14,304; the salicylates, such as salicylic acid, aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepiract, clidanac, oxepinac, and felbinac; the fenamates, such as mefenamic, meclofenamic, flufenamic, niflumic, and tolfenamic acids; the propionic acid derivates, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and the pyrazoles, such as phenybutazone, oxyphenbutazone, feprazone, azapropazone, and trimethazone.

Antimicrobials may be, but are not limited to, antibiotics (amikacin, gentamycin, neomycin, tobramycin, kanamycin, meropenem, imipenem, cefaclor), antivirals (abacavir, aciclovir, amantadine, boceprevir, cidofovir, darunavir, edoxudine, famciclovir, ganciclovir, imunovir, inosine, interferon, lamivudine, nexavir, oseltamivir, penciclovir, ribavirin, rimantadine, viramidine, zidovudine) and antifungals (Miconazole, ketoconazole, itraconazole, clotrimazole, econazole, fluconazole, voriconazole, abafungin, naftifine, caspofungin, micafungin, benzoic acid, griseofulvin).

Anti-inflammatory drugs may be, but are not limited to, zinc salts, including zinc salts of polysaccharide acids, such as hyaluronic acid.

In one embodiment of the instant invention, the polyanionic biopolymer solution and/or the solution comprising at least one divalent cation provided in the present invention may further comprise a polysaccharide.

In one embodiment of the instant invention, the polyanionic biopolymer solution and/or the solution comprising at least one divalent cation provided in the present invention may further comprise a protein and/or a peptide, e.g. an adhesion protein, a granulocyte-colony stimulating factor, erythropoietin, bone morphogenic protein, or tissue plasminogen activator.

In one further embodiment of the present invention, the polyanionic biopolymer solution and/or the solution comprising at least one divalent cation provided in the present invention may further comprise one or more pharmaceutical excipients selected from antioxidants, viscosity enhancers/modifiers, hydrating agents, bulking substances, tonicity agents, preservatives and surface active agents, or a mixture thereof.

Antioxidants may be, but are not limited to, vitamin E, vitamin C, glutathione, coenzyme Q, resveratrol, bisulfite sodium, butylated hydroxyl anisole/toluene, cysteinate, dithionite sodium, gentisic acid, glutamate, formaldehyde sulfoxylate sodium, metabisulfite sodium, monothiogylcerol, propyl gallate, sulfite sodium, thiogycolate sodium, flavonoids, catalase, lycopene, carotenes, lutein, superoxide dismutase and peroxidases or mixtures thereof.

Viscosity enhancers may be, but are not limited to, glycerol, xanthan gum, polyethylene glycol (PEG), alginate, carbomers, cellulose derivatives, dextrans, and carrageenan, starches, gum, acacia, tragacanth, gelatin, polyvinylpyrrolidone, albumin, dextran or mixtures thereof.

Bulking substances or tonicity modifiers may be substances such as glycerol, lactose, mannitol, dextrose, sodium or potassium chloride, sodium sulphate and sorbitol, in general at a concentration up to 5% depending upon the chosen substance.

Surface active agents may be, but are not limited to, polysorbate 20, polysorbate 80, polysorbate 40, polysorbate 60, polysorbate 65, Pluronic F68, Cetrimoniumbromid, Cetylpyridiniumchlorid, Brij 72, Brij 30, Brij 35, deoxycholate, lecithine, egg phospholipids, soy phospholipids, tocopheryl polyethylene glycol succinate or mixtures thereof.

In particular embodiments, the polyanionic biopolymer solution and/or the solution comprising at least one divalent cation provided in the present invention may further comprise a polysaccharide.

In one embodiment, the polysaccharide is hyaluronic acid and/or salts thereof.

The stability of the polymer solution is at least 12 months, more particularly at least 24 months, and most particularly at least 36 months. In certain embodiments, the present invention provides a method for the in situ preparation of a filler for aesthetic purposes.

In a particular embodiment, the present invention provides a method for the in situ preparation of a dermal filler, particularly for a non-medical treatment and/or use of a dermal filler.

In a particular embodiment, the dermal filler according to the instant invention is for the treatment of, or for the use in the treatment of, wrinkles and/or folds.

Wrinkles that may be treated by employing the method according to the instant invention include, but are not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles, and acne scars.

In another embodiment of the present invention, the polyanionic biopolymer as defined according to the instant invention is used for the treatment of, or for the use in the treatment of, a medical condition, including lipoatrophy, vocal fold insufficiency, gastroesophageal reflux disease (GERD), urine incontinence, vesico ureteral reflux (VUR), and the treatment of a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles and acne scars.

In another embodiment of the present invention, the polyanionic biopolymer as defined according to the instant invention is used in, or for the use in, aesthetic, plastic, cosmetic, dental or general surgery, in ophthalmology, in otology, in otorhinolaryngology, in orthopedics, for preventing tissue adhesions, or in urology.

The present invention further pertains to methods of using the polyanionic biopolymer as defined according to the present invention for aesthetic purposes, including the use as dermal filler, such as in the treatment of wrinkles and/or folds, particularly for a non-medical treatment and/or use of a dermal filler.

In another embodiment, the tissue augmentation effect lasts at least for three months, more particularly at least 6 months, and most particularly at least 12 months.

The present invention further pertains to methods of using the polyanionic biopolymer as defined according to the instant invention, and optionally, the solution comprising at least one divalent cation, for the therapeutic treatment of a patient in need thereof, such as in the treatment of lipoatrophy, vocal fold insufficiency, gastroesophageal reflux disease (GERD), urine incontinence, vesico ureteral reflux (VUR), and the treatment of a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet and perioral wrinkles.

The present invention further pertains to a method of treating a medical condition, including lipoatrophy, vocal fold insufficiency, gastroesophageal reflux disease (GERD), urine incontinence, vesico ureteral reflux (VUR), or a psychological condition caused by the appearance of an aesthetic deficiency, including, but not limited to, frown lines, medium depth wrinkles, such as nasolabial folds, lip augmentation, forehead wrinkles, glabellar lines, obvious mild to moderate nasal furrows and cheek wrinkles, crow's feet, perioral wrinkles and acne scars, wherein said method comprises a step of administering a polyanionic biopolymer as defined according to the instant invention, and optionally, the solution comprising at least one divalent cation, to a patient in need thereof

The present invention further pertains to a method of using a polyanionic biopolymer as defined according to the instant invention, and optionally, the solution comprising at least one divalent cation, according to the present invention in aesthetic, plastic, cosmetic, dental or general surgery, in ophthalmology, in otology, in otorhinolaryngology, in orthopedics, as products for preventing tissue adhesions, or in urology, wherein said method comprises a step of administering a polyanionic biopolymer as defined according to the instant invention, and optionally, the solution comprising at least one divalent cation, to a patient in need thereof.

In one further embodiment, the concentration of the polyanionic biopolymer is between 0.1 and 5.0 wt-% relative to the total weight of the solution.

In one further embodiment, where the method further comprises the step of applying a solution comprising at least one divalent cation to the application site before or after the application of the polymer solution, the amount of the at least one divalent cation is calculated in a way that up to 0.5 divalent cations, particularly between 0.4 and 0.5 divalent cations, per 1 carboxylate group in the polyanionic biopolymer are applied to the application site.

In one embodiment, the pH of the polyanionic biopolymer solution and/or the solution containing divalent cations is adjusted to a value between 7.0 and 7.8, particularly between 7.2 and 7.6, more particularly the pH value is 7.4.

In another embodiment, the viscosity of the polyanionic biopolymer solution is in the range from about 10 mPa*s to about 500 mPa*s measured by the falling ball viscometer.

In another embodiment, the in situ gelling effect determined by the change of viscosity after the in situ cross-linking is an increase of viscosity at least more than 50% of the initial viscosity of the polymer solution, more particular at least more than 100% of the initial viscosity and most particular at least more than 200% of the initial viscosity (see Example 3 below).

In another embodiment, the syringeability determined by the extrusion force of the polymer solution through a 27G needle is in the range of 1-30 N, particular between 1-20 N and more particular the extrusion force of the polymer solution through a 30G needle is between 1-25 N.

The polyanionic biopolymer employed in the method provided in the present invention may be mixed with other polymers or biopolymers of natural or synthetic origin in order to modify their physical properties. In one embodiment, the polyanionic biopolymer is pectin mixed with hyaluronic acid. In another embodiment, the polyanionic biopolymer is gellan mixed with hyaluronic acid. This may be achieved by preparing a solution having 0.5 wt-% based on the total weight of the composition hyaluronic acid and 0.5 wt-% based on the total weight of the composition gellan gum. As a result, a filler comprising gellan gum-hyaluronic acid is obtained.

The polyanionic biopolymer employed in the method provided in the present invention may be mixed with polymers in order to modify their physical properties. In one embodiment, the polyanionic biopolymer is pectin mixed with gellan gum. In another embodiment, the polyanionic biopolymer is gellan gum mixed with pectin.

According to the process of the present invention the aqueous solution comprising the divalent cation may comprise a combination of different divalent cations. In one embodiment, the aqueous solution comprising the divalent cations comprises a combination of copper and zinc. In one further embodiment, the aqueous solution comprising the divalent cations comprises a combination of calcium and zinc. In another embodiment, the aqueous solution comprising the divalent cations comprises a combination of barium and calcium.

The present invention further pertains to a kit comprising (i) a polyanionic biopolymer solution as defined according to the present invention; and (ii) a solution comprising at least one divalent cation as defined according to the present invention; and (iii) optionally, one or more injection devices. In one embodiment, the injection device comprises a 25- to 32-gauge needle. The size of the needle will be determined by the solution composition, the depth of the injection site and the injection volume. In certain embodiments, the injection device is disposable. In one embodiment, the injection device is made of sterile glass.

The present invention further pertains to an injection device comprising a solution as disclosed herein. In one embodiment, the injection device comprises a 25- to 32-gauge needle. The size of the needle will be determined by the solution composition, the depth of the injection site and the injection volume. In certain embodiments, the injection device is disposable. In one embodiment, the injection device is made of sterile glass.

In one embodiment, the injection device and the solutions provided herein are both sterile and non-pyrogenic e.g. containing less than 10 EU (Endotoxin Unit, a standard measure) per dose or application. The methods of achieving the sterility of the solutions are those known to the person skilled in the art.

Isotonicity of the filler may be accomplished by employing sodium chloride, glycerol, or other pharmaceutically acceptable agents such as dextrose.

A pharmaceutically acceptable preservative may be employed to improve the shelf-life of the solutions. The preservative may be, but is not limited to, benzalkonium chloride, thiomersal, parabens, chlorobutanol, benzethonium chloride, m-cresol, phenol, 2-phenoxyethanol, phenyl mercuric nitrate or benzyl alcohol. The suitable concentration of the preservative agent is between about 0.001% to 5% based on the total weight of the composition and the agent selected.

In another embodiment, the injection volume of the solutions is between 0.1 and 100 ml, particular between 0.1 and 50 ml, more particular between 0.1 and 30, 0.1 and 20, or 0.1 and 10 ml, and most particular between 0.1 and 5, 0.1 and 2, or 0.1 and 1 ml. Alternatively, the volume can be higher than 100 ml if larger areas are augmented.

In another aspect, the invention relates to a method, wherein the in situ formed fillers are redissolved after implantation by injecting chelating agents into the tissue, like EDTA, citrate, pentetic acid, diethylene triamine pentaacetic acid, 2,3-dimercapto-1-propanesulfonic acid and/or salts thereof.

The invention is now described with reference to the following examples. These examples are provided for the purpose of illustration only and the invention should not be construed as being limited to these examples, but rather should be construed to encompass any and all variations which become evident as a result of the teaching provided herein. The following materials and methods are provided with respect to the subsequent examples but do not limit a multiplicity of materials and methodologies encompassed by the present invention.

### EXAMPLES

### Example 1

### In situ Formation of a Filler Without Additional Divalent Cations

A tissue augmentation effect is achieved by the application of 1 ml of a solution containing 2% pectin (degree of esterification is 24%, degree of amidation is 25% and galacturonic acid content is 91%) in PBS at pH 7.0. A gelling effect is recognized after 48 h of gelling.

### Example 2

### In situ Formation of a Filler with Additional Divalent Cation

A 2 ml injectable solution containing 10 mM calcium ions is initially injected into the area for tissue augmentation. In a next step the tissue augmentation effect is achieved by the application of 1 ml of a solution containing 2% pectin (degree of esterification is 33%, degree of amidation is 0% and galacturonic acid content is 85%) in PBS at pH 7.0. The tissue augmentation effect occurs within 6 h.

### Example 3

### In situ Formation of Polymer Solution with Media Which Mimics the Physiological Electrolyte Concentration of the Extracellular Tissue

2.0% Pectin solution 1: Degree of esterification is 33%, degree of amidation is 0% and galacturonic acid content is 85%

2.0% Pectin solution 2: Degree of esterification is 24%, degree of amidation is 25% and galacturonic acid content is 91%.

Example 3 shows the superior gelling properties of pectin in contrast to alginate. Viscosity of a pectin solution increases when the solution is mixed with Ringer solution in a 1:1 ratio. The Ca²⁺ ion concentration in the Ringer solution is sufficient to initiate the gelling of the polymer. In contrast, when alginate is mixed with Ringer solution, no increase in viscosity was observed. Even the viscosity declined due to the dilution of the alginate solution. Gellan gum also show an increase in viscosity in presence of Ringer solution. Results for gellan gum are not shown, since the viscosity of the gellan gum solution mixed with Ringer solution was too high to be determined by the falling ball viscometer.

### Example 4

### Viscosity of Polymer Solution

Another advantage of pectin and gellan gum is its low viscosity of the polymer solution in contrast to alginate. Therefore lower injection forces are necessary and smaller gauge needles can be used to eject the filler and thus a better handling and administration and patient compliance is achieved when using pectin and gellan gum as filler material.

### Comparative Example 5

### Experiments for the in situ Formation of Fillers Using Alternative Polyanionic Biopolymers and Polymers

In addition to the work with pectin, gellan and alginate shown in Examples 1 to 4, the following alternative polyanionic biopolymers and polymers were tested for their performance in the preparation of in situ fillers: Xanthan; Carragenan; Gum arabic; Guar gum; Karakya; Fucoidan; polyacrylic acids; carboxymethyl cellulose; polymethacrylates; hyaluronic acid; Ceratonia siliqua; Tragacanth.

However, in contrast to pectin and gellan, none of the alternative polymers resulted in the formation of gels under in situ conditions that would have enabled their use as an in situ filler.

1-12. (canceled)

13. A method for the in situ preparation of a filler, comprising the step of applying a polyanionic biopolymer solution at an application site below the dermis of a patient for restoring volume at the application site, wherein the polyanionic biopolymer is selected from pectin and gellan.

14. The method according to claim 13, further comprising the step of applying a solution comprising at least one divalent cation to the application site before or after the application of the polymer solution.)

15. The method of according to claim 14, wherein the at least one divalent cation is selected from barium, zinc, copper, calcium and magnesium, and mixtures thereof.

16. The method according to claim 13, wherein the polyanionic biopolymer is pectin.

17. The method according to claim 16, wherein:
a. the pectin has a degree of amidation from about 0% to about 30%;
b. the pectin has a degree of esterification from about 0% to about 60%;
the pectin has a molecular weight distribution from about 50 to about 6000 kDa; and/or
d. the at least one divalent cation is selected from calcium, barium, zinc and copper, and mixtures thereof.)

18. The method according to claim 13, wherein the polyanionic biopolymer is gellan.

19. The method according to claim 18, wherein:
a. the gellan has a molecular weight distribution from about 50 to about 5000 kDa; and/or
b. the at least one divalent cation is selected from copper, calcium, zinc and mixtures thereof.

20. The method according to claim 14, wherein the polyanionic biopolymer solution and/or said solution comprising at least one divalent cation further comprise(s) one or more active pharmaceutical ingredients selected from anesthetics, analgesics, anti-microtias, anti-inflammatory drugs, growth factors, hormones, cosmeceuticals, vitamins, nutrients, stimulants, steroids, vasoconstrictors, anti-thrombotic agents, anti-coagulation agents, tranquilizers, muscle relaxants, antifungals, lipolytic agents and biorejunevation agents.

21. The method according to claim 14, wherein the polyanionic biopolymer solution and/or the solution comprising at least one divalent cation further comprise(s) one or more pharmaceutical excipients selected from antioxidants, viscosity enhancers/modifiers, hydrating agents, bulking substances, tonicity agents, preservatives and surface active agents, and mixtures thereof.)

22. The method according to claim 14, wherein the polyanionic biopolymer solution and/or the solution comprising at least one divalent cation further comprise(s) a polysaccharide.

23. The method according to claim 23, wherein the polysaccharide is hyaluronic acid and/or salts thereof.

24. The method according to claim 13, wherein the filler is an aesthetic filler.

25. The method according to claim 24, wherein the aesthetic filler a dermal filler.

26. The method according to claim 25, wherein the dermal filler is for the treatment of wrinkles and/or folds.

27. A method for treating a medical condition selected from: lipoatrophy, vocal fold insufficiency, gastroesophageal reflux disease (GERD), urine incontinence, vesico ureteral reflux (VUR), and a psychological condition caused by the appearance of an aesthetic deficiency, in a subject in need thereof, comprising administration of a polyanionic biopolymer selected from pectin and gellan.

28. A polyanionic biopolymer as defined according to claims 13 for use in aesthetic, plastic, cosmetic, dental or general surgery, in ophthalmology, in otology, in otorhinolaryngology, in orthopedics, in preventing tissue adhesions, or in urology.

29. A kit comprising (i) a polyanionic biopolymer solution wherein the polyanionic biopolymer selected from pectin and gellan; and (ii) a solution comprising at least one divalent cation.
